(19) United States
(12) Patent Application Publication
(10) Pub. No.: US 2005/0096541 A1
(43) Pub. Date: May 5, 2005

(54) Panniculus measuring apparatus using ultrasound

(75) Inventor: Osamu Fukuda, Ibaraki (JP)

Correspondence Address: BAKER & BOTTS, 30 ROCKEFELLER PLAZA, NEW YORK, NY, 10112, US

(73) Assignee: National Institute of Advanced Industrial Science and Technology, Tokyo (JP)

(21) Appl. No.: 10/977,607

(22) Filed: Oct. 29, 2004

(30) Foreign Application Priority Data
Oct. 29, 2003 (JP) 2003-368686
Oct. 13, 2004 (JP) 2004-298531

(51) Int. Cl. A61B 8/00

(52) U.S. Cl. 600/437; 600/447

(57) ABSTRACT

The present invention provides a small, light, easy to handle and low-price apparatus which allows measurement or quantification of panniculus in a mode of use using a personal computer not only at specific facilities such as a medical institution but also at home. The present invention is constructed of a small, light ultrasonic probe, an ultrasonic oscillation element drive/detection circuit connected to ultrasonic oscillation elements housed in the probe through a multiplexer, a focusing circuit which makes an echo image transmitted through a reflected signal of ultrasound obtained from the detection circuit visually well defined by means of digital delays, a data communication circuit which transfers image information to the personal computer and transmits a control signal from the personal computer to a control circuit, a control circuit which controls the ultrasonic oscillation element drive/detection circuit, focusing circuit and data communication circuit, and measuring software which constitutes an interface of the apparatus on the personal computer. Of these components, components from the drive/detection circuit to the data communication circuit are housed in the measuring apparatus.

### BACKGROUND OF THE INVENTION

1. Field of the Invention

The present invention relates to a fat measuring apparatus using ultrasound, capable of easily measuring/quantifying panniculus with a high degree of accuracy, used by being connected to a personal computer without mounting an operation panel which serves as a display or interface in the main unit of the apparatus, with an ultrasonic oscillation element connected to a drive/detection circuit through a multiplexer to realize size, weight and cost reductions of the overall circuit and probe, and further using a technology capable of obtaining high-definition images with focus achieved on an entire measuring region using a digital delay circuit.

2. Background Art

Enabling fat, muscle and bone of body tissue to be visualized and presented to the user is quite significant from the standpoints of health care to prevent life style related diseases, exercise management of athletes or beautification, etc.

In response to this demand, there are apparatuses designed to measure fat using the principles of a medical ultrasound diagnostic imaging apparatus (e.g., JP Patent Publication (Kokai) No. 2003-235848A) and apparatuses currently on the market, but these apparatuses implement all of a probe, measuring circuit, display circuit, interface, etc., on a single apparatus, which makes it difficult to drastically reduce the size or reduce manufacturing cost.

Interfaces with external apparatuses of a conventional fat gauge are limited to output of video signals, printer output or transfer of still images via a communication cable and are unable to even accept command and parameter inputs from the outside.

### SUMMARY OF THE INVENTION

The present invention has been implemented taking into account the problems described above and it is an object of the present invention to provide a small, light, easy to handle and low-price apparatus for panniculus measurement/quantification so as to be used not only in specific facilities such as a medical institution but also at home, etc., in a mode of use using a personal computer.

The present invention makes it possible to use an all-purpose interface (USB, etc.) capable of high-speed transfer of image data so as to allow connection of a personal computer currently on the market, use a light housing and cables so as to capitalize on the effectiveness of a small and light circuit, record data in a hard disk of the computer so as to make the most of a merit of using a personal computer as an interface with the user and improve operability by making an interface screen interactive like a bank ATM apparatus.

In order to attain the above described object, the present invention provides a panniculus measuring apparatus using ultrasound, comprising a small, light ultrasonic probe, an ultrasonic oscillation element drive/detection circuit connected to ultrasonic oscillation elements housed in the probe through a multiplexer, a focusing circuit which makes an echo image transmitted through a reflected signal of ultrasound obtained from the detection circuit visually well-defined by means of digital delays, a data communication circuit which transfers image information to a personal computer and transfers a control signal from the personal computer to a control circuit, a control circuit which controls the ultrasonic oscillation element drive/detection circuit, focusing circuit and data communication circuit, and measuring software which constitutes an interface of the apparatus on the personal computer.

The panniculus measuring apparatus using ultrasound according to the present invention (1) is used by being connected to a personal computer to thereby separate an interface with the user and an image data display function, etc., from the main unit of the apparatus, (2) connects an ultrasonic oscillation element and a drive/detection circuit through a multiplexer and (3) uses a digital delay system for focusing to realize a plurality of focal points using a simple delay circuit to thereby reduce the size, weight and cost, and allow measurement of high definition image data.

### BRIEF DESCRIPTION OF THE DRAWINGS

FIG. 1 is a conceptual illustration of an apparatus;

FIG. 2 is a block diagram of a system;

FIG. 3 illustrates connectability of a plurality of probes;

FIG. 4 illustrates a method of connecting ultrasonic oscillation elements;

FIG. 5 shows a principle of focusing; and

FIG. 6 shows a structure of a focusing circuit.

### DETAILED DESCRIPTION OF THE PREFERRED EMBODIMENTS

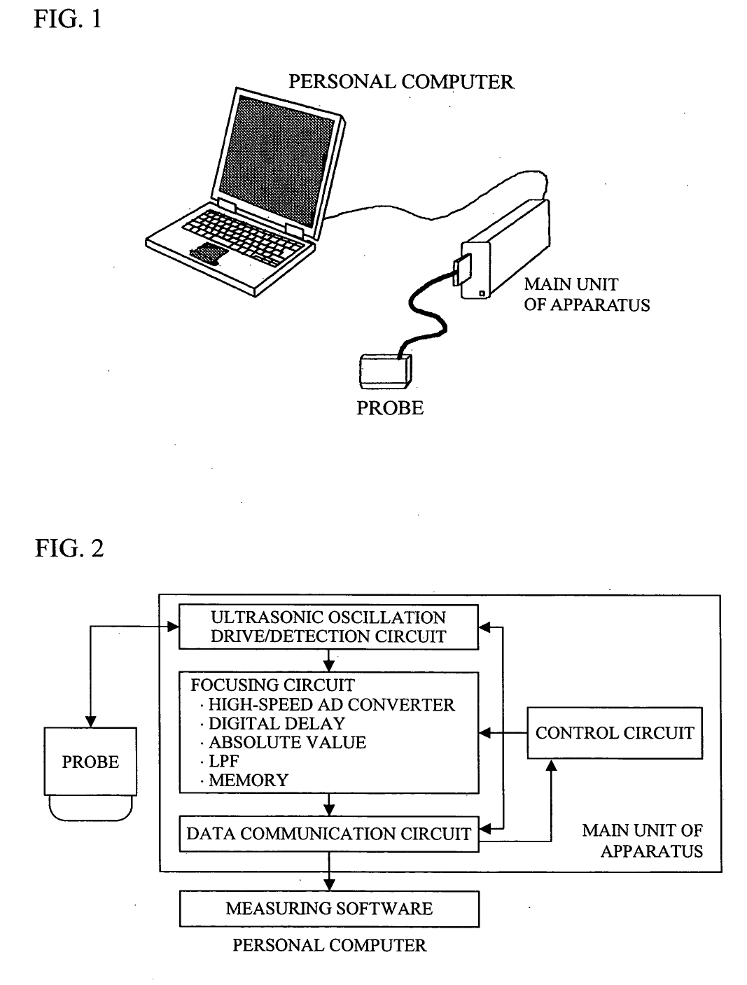

With reference now to the attached drawings, an embodiment of the present invention will be explained in detail. FIG. 1 is a conceptual illustration of an apparatus according to the present invention. As shown in the figure, the apparatus of the present invention is used by being connected to a personal computer and has a very light, compact outer appearance compared to a conventional medical diagnostic imaging apparatus. FIG. 2 is a block diagram schematically showing the system. As shown in this figure, the present system is constructed of a small, light ultrasonic probe, an ultrasonic oscillation element drive/detection circuit connected to an ultrasonic oscillation element housed in the probe through a multiplexer, a focusing circuit which makes an echo image transmitted through an ultrasound reflected signal obtained from a detection circuit a visually high definition image through a digital delay, a data communication circuit which transfers image information to a personal computer and transfers a control signal from the personal computer to a control circuit, a control circuit which controls the ultrasonic oscillation element drive/detection circuit, focusing circuit and data communication circuit, and measuring software which constitutes an interface of the apparatus on the personal computer. Of these components, the components from the drive/detection circuit to the data communication circuit are housed in the main unit of the measuring apparatus.

The apparatus of the present invention can be used by being connected to a personal computer, and can thereby omit an image display video circuit and operation interface (keyboard switch and LED, etc.), reduce the size and cost and transfer image data as digital information, and thereby provide images clearer than images recorded using a video capture system of a conventional apparatus.

Furthermore, the apparatus of the present invention can use various types of expandability provided for a personal computer. For example, it is possible to measure information from another diagnostic apparatus (impedance type fat gauge, force sensor, electromyograph, etc.) connected to the personal computer, combine the information with results obtained from this apparatus to realize comprehensive diagnosis, use the Internet, etc., to evaluate and manage the measured data at a clinic at a remote place.

The sections of the present apparatus will be explained more specifically below.

The following paragraphs have particular relevance to the probe.

Since a conventional fat gauge is supposed to be used with a probe held by hand, the fat gauge often has a roundish shape and has a width of range within which images can be actually measured approximately half the horizontal width of the housing of the probe, and therefore it has been impossible to couple a plurality of probes and measure a wide range or use another machine or apparatus (robot arm, slider, etc.) connected thereto.

On the other hand, the ultrasonic probe of the present invention is constructed to be small and light in a rectangular shape to facilitate stable attachment to a machine or apparatus (robot arm, movement slider, etc.) or having an attachment. Furthermore, the ultrasonic probe allows image taking over the entire range of the horizontal width of the surface of contact of the probe, couples a plurality of probes side by side and thereby allows images over a wide range to be measured.

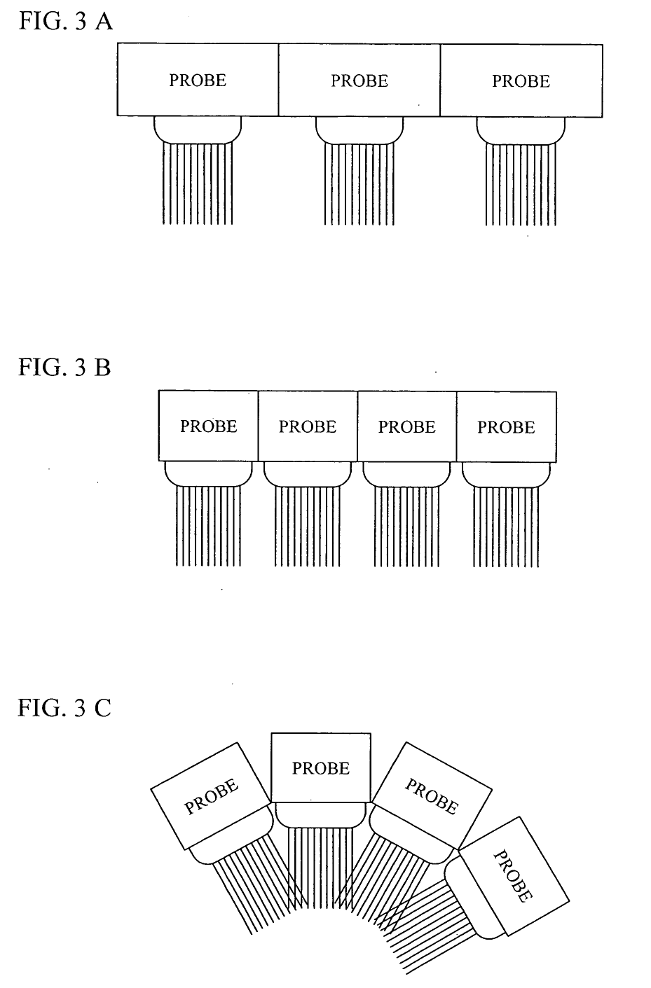

In this way, the probe reduces the size and extends the effective range of image taking up to substantially the full width of the probe to allow images over a wide range to be measured with a plurality of probes arranged as shown in FIG. 3. For example, in the case of FIG. 3A, the range in which image measurement is effective is narrow with respect to the horizontal width of the probe, and even if a plurality of probes are coupled to combine images, images obtained have a large proportion of missing parts, and therefore it is possible to reduce the proportion of the missing parts by extending the range effective for image taking as shown in FIG. 3B. Furthermore, arranging the probes in a curve shown in FIG. 3C allows the inside areas to be combined with relatively fewer missing parts.

The following paragraphs have particular relevance to the ultrasonic oscillation drive/detection circuit.

The probe in the conventional apparatus has many internal ultrasonic oscillation elements connected one to one with their respective drive/detection circuits, and therefore requires quite many circuits corresponding in number to elements and requires thick and heavy cables to connect those circuits.

For example, a standard existing probe has 64 ultrasound oscillators arranged and includes individual signal lines and a common line which combines those signal lines into one. Thus, the existing probe requires at a minimum of 65 cables.

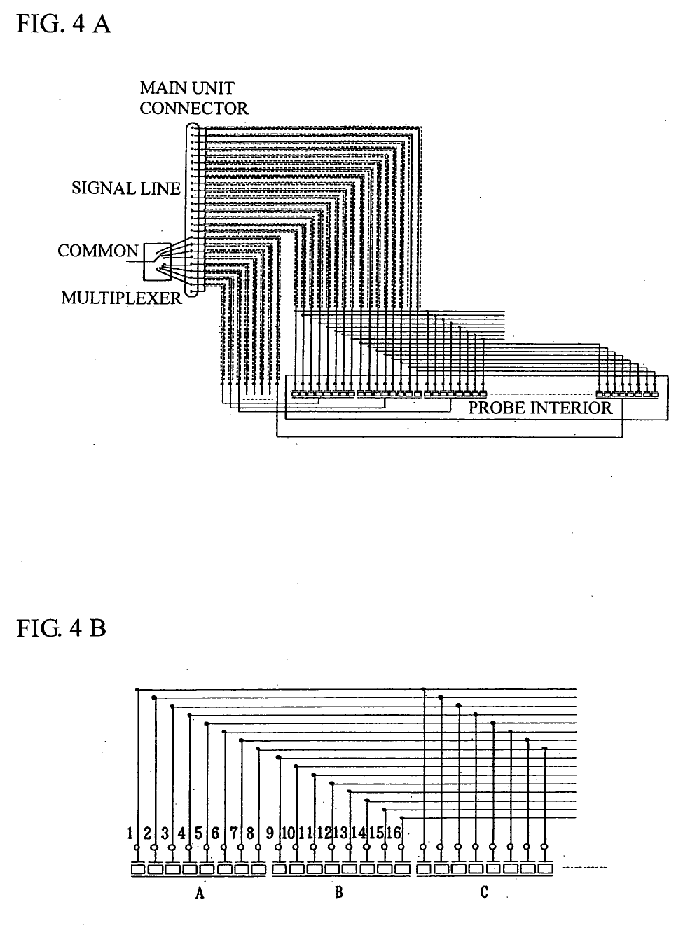

In contrast, by dividing a common line into 8 sets of 8 oscillation elements and thereby realizing multiplexing, it is possible to drastically reduce the number of lines as shown in FIG. 4A. In the case of this figure, all that is required is only 25 cables with 16 signal lines and 8 common lines connected, which reduces the diameter of cables, reduces the weight and reduces the cost of a connector as well.

FIG. 4B illustrates the method of scanning oscillation elements. Suppose the oscillation elements perform drive/detection operations in groups of eight. First, signal lines 1, 2, 3, 4, 5, 6, 7, 8 and a common line A are used. The eight elements to be used next are a group which corresponds to all the eight elements shifted by one element horizontally and signal lines 2, 3, 4, 5, 6, 7, 8, 9 and the common lines A (corresponds to the signal lines 2 to 8) and B (corresponds to the signal line 9) are used. As shown above, using eight signal lines and one or two common lines makes it possible to repeat scanning while shifting eight oscillators one at a time.

An example of the technique for connecting a number of ultrasonic oscillation elements to a smaller number of drive/detection circuits is a circuit configuration utilizing a multiplexer. By using a multiplexer, the number of drive/detection circuits can be reduced.

However, in this case, there is a tradeoff between the “number of drive/detection circuits” and the “number of channels of signal lines switched by the multiplexer.” This means that as the number of the drive/detection circuits is reduced, the number of channels of the signal lines switched by the multiplexer, i.e., the number of circuits to be switched, increases. Furthermore, if the multiplexer circuit is located not inside the probe but near the drive/detection circuits, there would no change in the number of channels of the cables connecting the oscillator in the probe and the drive/detection circuits, nor in the number of the pins of the connectors connected to the cables.

In the case where a plurality of oscillators are switched using a multiplexer, it is desirable to perform the switching in a simple, fast and stable manner in order to capture clear moving images with no noise at high speed. Generally, when controlling the drive/detection of a plurality of oscillators and the beam forming using a multiplexer, the following two steps, namely: 1) switch the multiplexer depending on the oscillator used, and 2) control the drive/detection circuits, must be performed each time. These two steps pose a problem in obtaining clear moving images and they also take time for processing.

FIG. 4 shows a configuration of a multiplexer designed to simultaneously reduce the number of drive/detection circuits, the number of channels of signal lines switched by the multiplexer, the number of cables, and the number of pins of connectors. The switching method employed is simple, fast, and stable and is therefore suitable for capturing clear moving images at high speed.

In the multiplexer of FIG. 4, the common lines connected to a plurality of ultrasonic oscillation elements contained in a probe are short-circuited from one end for each number of oscillation elements used for a single instance of beam-forming transmission/reception. Also, with regard to these sets of short-circuited oscillators, for each of the odd-numbered groups or even-numbered groups from the end of the probe, the signal lines connected to the oscillators located at the same position in each set are short-circuited. In this case, it is possible to carry out beam forming for driving/detection using a set of multiple oscillators, and it is also possible to perform scanning for ultrasonic imaging by displacing the set of oscillators one at a time. Although FIG. 4 shows 64 probe elements and indicates that the number of oscillators used for a single beam forming is eight, these numbers are merely exemplary.

In the case of the example of FIG. 4, in which the number of probe elements is 64 and the number of oscillators used for a single beam forming is eight, with regard to the common lines, (1, 2, 3, 4, 5, 6, 7, 8), (9, 10, 11, 12, 13, 14, 15, 16), (17, 18, 19, 20, 21, 22, 23, 24), (25, 26, 27, 28, 29, 30, 31, 32), (33, 34, 35, 36, 37, 38, 39, 40), (41, 42, 43, 44, 45, 46, 47, 48), (49, 50, 51, 52, 53, 54, 55, 56), (57, 58, 59, 60, 61, 62, 63, 64) are short-circuited, and, with regard to the signal lines, (1, 17, 33, 49), (2, 18, 34, 50), (3, 19, 35, 51), (4, 20, 36, 52), (5, 21, 37, 53), (6, 22, 38, 54), (7, 23, 39, 55), (8, 24, 40, 56), (9, 25, 41, 57), (10, 26, 42, 58), (11, 27, 43, 59), (12, 28, 44, 60), (13, 29, 45, 61), (14, 30, 46, 62), (15, 31, 47, 63), (16, 32, 48, 64) are short-circuited. Note that the numbers in the parentheses indicate the positions of the oscillators counted from the end. In this manner of connection, the number of cables required, which has conventionally been 65, can be reduced to 24.

Hereafter the procedure for driving and detection in the ultrasonic probe is described with reference to FIG. 4, assuming that the oscillation elements perform the drive/detection operation in groups of eight. Initially, the signal lines 1, 2, 3, 4, 5, 6, 7, and 8 and a common line A (short-circuiting 1, 2, 3, 4, 5, 6, 7, and 8) are used. In this case, however, a common line B (short-circuiting 9, 10, 11, 12, 13, 14, 15, and 16) is also selected in advance in addition to common line A, and the multiplexer is switched such that the two common lines A and B can be used. The eight elements that are used next are the group of elements that have been displaced by one element, such that the signal lines are 2, 3, 4, 5, 6, 7, 8, and 9, with the common lines A and B being used. As mentioned above, since common lines A and B are selected in advance, noise due to the switching of the multiplexer can be reduced and a stable transmission/reception can be realized quickly by simply changing the signal lines used. Namely, because it is possible to select the common lines in advance, the drive/detection operation can be performed in one step.

When the signal lines used have reached 9, 10, 11, 12, 13, 14, 15, and 16, the common line used is changed from A to C (short-circuiting 17, 18, 19, 20, 21, 22, 23, and 24), such that B and C have been selected. In other words, the switching of the multiplexer is performed in advance. In this way, when the signal lines 10, 11, 12, 13, 14, 15, 16, and 17 are used, a stable transmission/reception can be realized quickly in one step. Thus, by using eight signal lines and one or two common lines, it becomes possible to repeat a stable scan quickly while displacing the eight oscillators one by one.

Thus, in accordance with the method of the invention, a multiplexer is used for reducing the number of drive/detection circuits. The multiplexer, however, is used only for the switching of the common lines and does not result in increasing the number of channels to be switched greatly. By partly short-circuiting the common lines and signal lines, the number of channels of cables and the number of pins of connectors can also be reduced. Furthermore, a transmission/reception beam forming can be performed using a plurality of oscillators on the basis of the switching of connection of the common lines used and of the combination of the signal lines used, while realizing the scan by displacing the set of oscillators one oscillator at a time. The switching is simple, fast, and stable, and is therefore suitable for capturing moving images clearly and at high speed.

While the existing product requires 64 switches for switching among elements to drive the ultrasonic oscillation elements, the apparatus in this example needs only 16 switches for switching among elements and eight common lines. Therefore, as opposed to 64 switches of the existing product, this apparatus requires 24 switches, and can thereby reduce the circuit scale and reduce cost as well.

In this way, the ultrasonic oscillation element drive/detection circuit is connected to many ultrasonic oscillation elements housed in the probe through a multiplexer and can control many elements through a fewer number of drive/detection circuits, and thereby construct a small, light apparatus at a lower cost.

The following paragraphs have particular relevance to the focusing circuit.

Normally, a focusing circuit of an ultrasound diagnostic apparatus adjusts focus to a desired position of an echo image by appropriately adjusting mutual timings of ultrasound signals received by many piezoelectric elements.

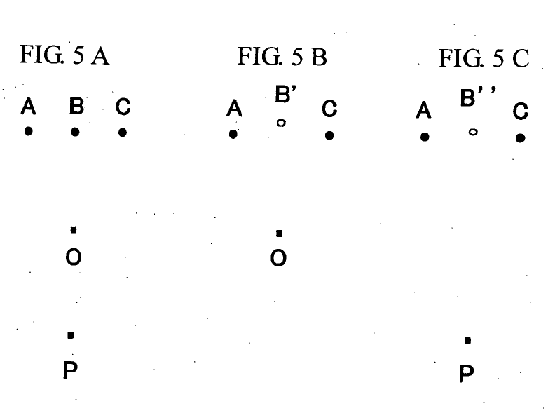

FIG. 5 is a schematic diagram of focusing. For example, in FIG. 5A, suppose O and P are sound sources (reflection sources) and A, B, C are sensors. Ultrasound waves emitted from O and P arrive at B earliest and arrive at A and C simultaneously with a small delay. At this time, the signal received by B is delayed by this time portion (delay time=OA-OB)÷sound velocity) and suppose as if the ultrasound wave had been received with a delay. Then, a relationship of OA=OB′=OC can be held as shown in (b). At this time, it is possible to obtain a sensor value triple the original signal by adding up the signals received at A, B′ and C.

Therefore, in FIG. 5B, focus is determined on point O. However, while a clear image can be obtained at focal point (O), the difference in the arrival time varies among different focal points (e.g., P in FIG. 5C), and therefore the relationship of OA=OB″=OC as shown in the figure cannot be held and the image becomes unclear.

To achieve correct focus for all actual images, it is necessary to change delay times depending on the distance between the sound source and sensor. The conventional ultrasound diagnostic apparatus normally uses an analog delay element to adjust this timing, but this delay time is determined to one delay time depending on the electrical characteristic of the element, and therefore the number of required elements increases cumulatively according to the number of points on an image to be focused, which increases the scale of the circuit.

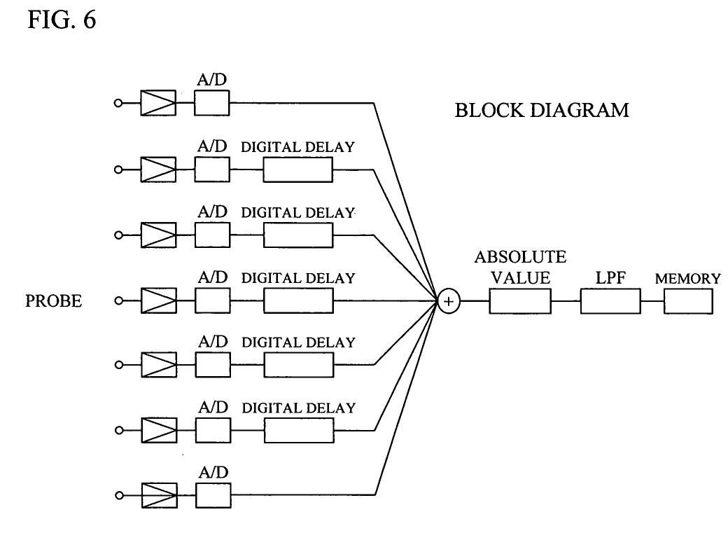

The apparatus in the example realizes such a delay using a digital circuit as shown in FIG. 6. Analog signals of their respective sensors are converted to digital signals by A/D converters and temporarily stored in shift registers and addresses at which data is read from these registers correspond to delay times. The minimum unit of delay time is determined by high-speed A/D conversion, shift register clocks.

As shown in FIG. 5(b), signals from a plurality of sensors are added up to obtain a strong signal and the data to be added in this case is selected from the shift registers. This data is read while changing addresses of the shift registers with time so as to achieve optimum focus. In this way, clear images in sharp focus are obtained from near to far ranges.

In this way, the focusing circuit converts reflected signals of ultrasound detected from many ultrasonic oscillation elements to digital signals through high-speed AD converters and shifts timings of the respective signals adequately by means of digital delays to continuously change focus positions of an echo image and achieve focus with high definition at the respective positions.

A signal resulting from adding up values from a plurality of sensors becomes a time-series signal which drastically changes between positive and negative with time. To obtain brightness information from this signal, as shown in FIG. 6, an amplitude component is picked up through absolute value processing, further passed through a low pass filter (LPF) to suppress time variations of drastic amplitude components, and brightness information easy to recognize visually is extracted and recorded in memory.

The following paragraphs have particular relevance to the data communication circuit.

In the apparatus in the example, a high-speed communication such as USB, LAN provided as standard for a personal computer is used for data communication with the main unit. An ultrasound image consists of 128 dots (H)×512 dots (V)×8 bits and has a data count of 64 Kbytes. When this data is communicated using USB1.1 (12 Mbps=1.5 Mbytes/sec), the transfer rate is 24 screens/sec. The ultrasound screen is displayed on the screen on the personal computer as real-time moving images. Using USB2.0 or 100baseLAN enables much faster transfers.

Thus, the data communication circuit transfers image data stored in memory to the personal computer as a time series of digital signals without any deterioration and makes it possible to flexibly set the size, resolution and transfer rate. The data communication circuit further receives a command related to parameters of the control circuit from the personal computer and can freely set the operating situation of the apparatus.

The following paragraph has particular relevance to the control circuit.

The control circuit receives parameters related to apparatus control through a communication circuit and controls the ultrasonic oscillation element drive/detection circuit, focusing circuit and data communication circuit according to the parameters.

The following paragraph has particular relevance to the measuring software.

The measuring means is constructed of measuring software. The measuring software plays the role of an interface with the operator and allows the operator to adjust parameters, etc., of the apparatus even when there is no operation panel, etc., in the main unit of the apparatus, enhances the convenience such as recording and browsing of data and parameters using a hard disk of the personal computer and allows image processing functions such as image data filtering, histogram adjustment and automatic quantification to be added as required through version upgrades of software.

The panniculus quantification apparatus using ultrasound of the present invention provides portability and can be used at home or in fields, and therefore the following industrial applications can be expected.

At cosmetic surgery, esthetic salon or home, this apparatus makes it possible to observe an amount of fat or muscle, balance between right and left and thereby determine the effects of diet, realize early discovery of rebound, evaluation and prevention, contributing to continuation of willingness of the customer, enhancement of persuasion effects about a prescription.

In the fields of professional, sports facilities, fitness club or sports medicine, this apparatus makes it possible to observe an amount of fat or muscle, balance between right and left and thereby observe the progress of training, evaluate fatigue, prognostication of injury, evaluate recovery, etc. Also when giving educational guidance, the apparatus enhances persuasion with a visual aid.

At plastic surgeon, institution for the aged, orthopedic clinic, chiropractics or home, this apparatus makes it possible to measure an amount of fat or muscle, measure the effects of rehabilitation and observe the progress, etc. Here, storing data allows evaluation by age, evaluation by constitution, evaluation of walking ability and ability of limbs.

While there have been described what are believed to be the preferred embodiments of the present invention, those skilled in the art will recognize that other and further changes and modifications may be made thereto without departing from the spirit of the invention, and it is intended to claim all such changes and modifications as fall within the true scope of the invention.

1. An ultrasonography apparatus, comprising:
an ultrasonic probe;
a multiplexer for switching between common lines to be used in a circuit constructed so as to short-circuit a common line connected to a plurality of ultrasonic oscillation elements housed in said probe every number of oscillators used for beam forming of one-time transmission and reception from one end and short-circuit between signal lines connected to oscillators at the same positions in every odd-numbered set or every even-numbered set of the plurality of short-circuited oscillators from the end of the probe;
an ultrasonic oscillation element drive and detection circuit connected to said multiplexer, which connects and switches between common lines used by this multiplexer, performs beam forming of transmission and reception by a plurality of oscillators according to a combination of signal lines used and performs scanning while shifting the set of oscillators one oscillator at a time; and
a unit for obtaining an echo image obtained from a reflected signal of ultrasound obtained from said detection circuit.

2. The ultrasonography apparatus of claim 1, wherein said ultrasonic probe has a rectangular shape to facilitate stable attachment to a machine or apparatus or has an attachment, enables images to be taken over the entire range of the horizontal width of the probe contact surface, and couples a plurality of probes side by side to measure images within a wide range.

3. The ultrasonography apparatus of claim 1, further comprising a focusing circuit which makes said echo image with visually high definition by a digital delay.

4. The ultrasonography apparatus of claim 1, further comprising a data communication circuit which transfers said echo image information to a personal computer and transmits a control signal from the personal computer to said apparatus.

5. The ultrasonography apparatus of claim 1, further comprising a control circuit which controls said ultrasonic oscillation element drive and detection circuit, focusing circuit and data communication circuit.

6. The ultrasonography apparatus of claim 1, wherein said focusing circuit converts reflected signals of the ultrasound detected from many ultrasonic oscillation elements to digital signals by high-speed AD converters, and adequately shifts timings of the respective signals by means of digital delays to thereby continuously change the focus positions of the echo image and make focusing at the respective positions well defined.

7. The ultrasonography apparatus of claim 1, wherein said data communication circuit transfers image data to the personal computer as a time series of digital signals without deterioration, flexibly sets the size, resolution and transfer rate, receives a command related to parameters of the control circuit from the personal computer and freely sets the operating state of the apparatus.

8. The ultrasonography apparatus of claim 1, wherein said control circuit receives parameters related to apparatus control through a communication circuit and controls the ultrasonic oscillation element drive and detection circuit, focusing circuit and data communication circuit according to the parameters.

9. The ultrasonography apparatus of claim 4, further comprising measuring means for constructing an interface of the apparatus on said personal computer, wherein said measuring means plays the role of an interface with the operator and allows the operator to adjust parameters of the apparatus even when there is no operation panel in the main unit of the apparatus, enhances the convenience such as recording and browsing of data and parameters using a hard disk of the personal computer and allows image processing functions such as image data filtering, histogram adjustment and automatic quantification to be added as required.

10. A panniculus measuring apparatus using ultrasound used by being connected to a personal computer, which constructs an interface on the personal computer to separate an interface with the user or an image data display function from the main unit of the apparatus, comprising:
an ultrasonic probe;
a multiplexer for switching between common lines to be used in a circuit constructed so as to short-circuit a common line connected to a plurality of ultrasonic oscillation elements housed in said probe every number of oscillators used for beam forming of one-time transmission and reception from one end and short-circuit between signal lines connected to oscillators at the same positions in every odd-numbered set or every even-numbered set of the plurality of short-circuited oscillators from the end of the probe;
an ultrasonic oscillation element drive and detection circuit connected to said multiplexer, which connects and switches between common lines used by this multiplexer, performs beam forming of transmission and reception by a plurality of oscillators according to a combination of signal lines used and performs scanning while shifting the set of oscillators one oscillator at a time;
a focusing circuit which makes an echo image obtained from a reflected signal of ultrasound obtained from said detection circuit an image with visually high definition by a digital delay;
a data communication circuit which transfers the echo image information to the personal computer and transmits a control signal from the personal computer to the control circuit; and
a control circuit which controls said ultrasonic oscillation element drive and detection circuit, focusing circuit and data communication circuit.

11. The panniculus measuring apparatus of claim 10, wherein said ultrasonic probe has a rectangular shape to facilitate stable attachment to a machine or apparatus or has an attachment, enables images to be taken over the entire range of the horizontal width of the probe contact surface, and couples a plurality of probes side by side to measure images within a wide range.

12. The panniculus measuring apparatus of claim 10, wherein said focusing circuit converts reflected signals of the ultrasound detected from many ultrasonic oscillation elements to digital signals by high-speed AD converters, and adequately shifts timings of the respective signals by means of digital delays to thereby continuously change the focus positions of the echo image and make focusing at the respective positions well defined.

13. The panniculus measuring apparatus of claim 10, wherein said data communication circuit transfers image data to the personal computer as a time series of digital signals without deterioration, flexibly sets the size, resolution and transfer rate, receives a command related to parameters of the control circuit from the personal computer and freely sets the operating state of the apparatus.

14. The panniculus measuring apparatus of claim 10, wherein said control circuit receives parameters related to apparatus control through a communication circuit and controls the ultrasonic oscillation element drive and detection circuit, focusing circuit and data communication circuit according to the parameters.

15. The panniculus measuring apparatus of claim 10, further comprising measuring means for constructing an interface of the apparatus on said personal computer, wherein said measuring means plays the role of an interface with the operator and allows the operator to adjust parameters of the apparatus even when there is no operation panel in the main unit of the apparatus, enhances the convenience such as recording and browsing of data and parameters using a hard disk of the personal computer and allows image processing functions such as image data filtering, histogram adjustment and automatic quantification to be added as required.
